(19) United States
(12) Patent Application Publication
(10) Pub. No.: US 2005/0106188 A1
(43) Pub. Date: May 19, 2005

(54) Dosage form for the oral administration of active ingredients, vitamins and/or nutrients

(76) Inventors: Rasoul Sedaghat Kerdar, Aachen (DE); Johannes Barthlomaus, Aachen (DE); Norbert Lamm, Roetgen (DE)

Correspondence Address: PERMAN & GREEN, 425 POST ROAD, FAIRFIELD, CT, 06824, US

(21) Appl. No.: 11/022,526

(22) Filed: Dec. 23, 2004

(30) Foreign Application Priority Data
Jun. 24, 2002 (DE) 102 28 192.0

(51) Int. Cl. A61K 9/00; E03B 9/20; B05B 12/14; A61M 31/00

(52) U.S. Cl. 424/400; 239/24; 604/500

(57) ABSTRACT

The invention relates to an administration form for orally administering active substances, vitamins and/or nutrients, including a drinking straw which is provided with a closing device that is made of a physiologically unproblematic material, the material being at least partly soluble in a conveying liquid and at least partly air-permeable, and a kit including the administration form and a conveying liquid.

The invention relates to a dosage form for the oral administration of active ingredients, vitamins and/or nutrients comprising a drinking straw containing an air-permeable closing device made from a physiologically unproblematic material which is at least partially soluble in or permeable to the conveying liquid and an active ingredient, vitamin and nutrient formulation, and to a kit comprising the dosage form and a conveying liquid.

It is known to administer active ingredients, vitamins and/or nutrients with the assistance of a drinking straw which contains an active ingredient, vitamin and/or nutrient formulation which is conveyable by a liquid. Patients who have difficulty in or an aversion to taking capsules or tablets are provided in this manner with a further dosage form for active ingredients, vitamins or nutrients.

The drinking straws described in the prior art for the oral administration of active ingredients, vitamins and/or nutrients comprise bungs or caps as a closing device in order to retain the active ingredient, vitamin and/or nutrient formulation located in the drinking straw. The bungs or caps must be removed before the drinking straw is used. There is a risk of spilling the active ingredient, vitamin and/or nutrient formulation while said closing devices are being removed.

The object therefore arose of providing a dosage form for the oral administration of active ingredients, vitamins and/or nutrients comprising a drinking straw with a closing device, which dosage form is as simple as possible to produce and does not exhibit the disadvantages of the dosage forms described in the prior art.

This object is achieved according to the invention by the provision of a dosage form for the oral administration of active ingredients, vitamins and/or nutrients comprising a drinking straw, which comprises two orifices, and which contains in the drinking straw a barrier device which is impermeable to an active ingredient, vitamin and/or nutrient formulation, but is permeable to air and a conveying liquid, a closing device which is impermeable to the active ingredient, vitamin and/or nutrient formulation prior to administration, and the active ingredient, vitamin and/or nutrient formulation arranged between the barrier device and the closing device, wherein the closing device is permeable to air, is soluble in the conveying liquid or permeable to the conveying liquid and consists of physiologically unproblematic material.

The closing device preferably very rapidly dissolves completely in the conveying liquid.

The closing device may take the form of a plug, a mesh or a film.

The closing device is preferably arranged between the barrier device (2) and the drinking straw orifice (8) from which the formulation is administered. The formulation is arranged between the barrier device (2) and the closing device (3). The closing device may be arranged in the drinking straw or directly closes the drinking straw orifice which serves as a mouthpiece.

The closing device preferably consists of at least one material or at least one compound selected from among the group comprising sugars and/or sugar-like compounds, preferably sucrose, glucose, fructose, mannitol, sorbitol, flucose, maltose, lactose, galactose, trehalose and/or sugar alcohols; cyclic sugars, preferably cyclodextrins; proteins, preferably gelatine, gelatine derivatives, gelatine hydroxylates; dextrins; proteins from soya, wheat and/or psyllium seeds; gums and/or gum-like materials, preferably agar, acacia, guar, xanthan; flour, preferably rye, wheat and/or rice flour; starch, preferably starch consisting of amylose with up to 6000 glucose units and amylopectin with 10-60 glucose units; modified starch, preferably starch with esterified hydroxy groups, hydroxyethyl starch, hydroxypropyl starch and/or distarch phosphates; pectins; dextrans; cellulose derivatives; preferably methylcellulose, ethylcellulose, hydroxymethylcellulose, hydroxyethylcellulose, carboxymethylcellulose or the alkali metal salts thereof; alginates, carrageenans; amino acid, preferably with 2-12 carbon atoms, particularly preferably glycine, L-alanine, L-aspartic acid, L-glutamic acid, L-hydroxyproline, L-leucine, L-isoleucine and/or L-phenylalanine, synthetic polymers, preferably polyvinylpyrrolidone; inorganic salts, preferably sodium phosphate, sodium chloride and/or aluminium silicate.

These materials and compounds may optionally also serve as a matrix material for immobilising the formulation.

A cotton wool-like plug is preferably used as the closing device, which plug has been produced from at least one compound or at least one material selected from among the group comprising sugars, preferably sucrose, glucose, fructose, mannitol, sorbitol, flucose, lactose and/or maltose; soluble cellulose material, preferably methylcellulose, ethylcellulose, hydroxymethylcellulose, hydroxyethylcellulose or an alkali metal salt of carboxymethylcellulose. The cotton wool-like material may be produced in accordance with a process described in U.S. Pat. No. 4,855,326, the corresponding disclosure consequently being introduced as a reference.

This plug preferably has a diameter which matches the internal diameter of the drinking straw.

The closing device at least partially dissolves on contact with the conveying liquid such that the latter can pass through unimpeded and entrain the formulation without being impeded when the formulation is taken. The barrier device preferably dissolves completely on contact with conveying liquid.

A flour wafer, which is also known as edible paper and is produced by baking, is also suitable as the closing device.

Water-soluble films made from physiologically unproblematic polymers such as polyvinyl alcohol or other materials from the above-stated group are also suitable as the closing device.

The closing device may also simultaneously serve as a matrix for active ingredients, vitamins and/or nutrients and/or the formulation thereof.

At least one end of the drinking straw may be provided with a removable cap, the mouthpiece of the dosage form preferably being so equipped.

The active ingredient, vitamin and/or nutrient formulation is preferably solid or pasty. A multiparticulate active ingredient, vitamin and/or nutrient formulation is preferred, wherein the particles of the multiparticulate formulation preferably have a diameter of 50-1500 μm.

The active ingredients, vitamins and/or nutrients per se or the overall active ingredient, vitamin and/or nutrient formulations may be insoluble or at least partially soluble in the conveying liquid.

If the active ingredients, vitamins or nutrients or the formulations thereof are soluble in the conveying liquid, they are dissolved by the conveying liquid as it flows past and entrained in dissolved form. If, on the other hand, the active ingredients, vitamins or nutrients or the formulations thereof are insoluble in the conveying liquid, they are suspended in the conveying liquid and entrained with the conveying liquid in suspended form. Multiparticulate formulations are preferably used for this purpose. The particles should be so small and light that they can be conveyed by the conveying liquid. Adhesion of the particles to one another or to the walls of the drinking straw should be as slight as possible, which may optionally be achieved by a suitable surface treatment of the particles.

Active ingredients, preferably therapeutic active ingredients, vitamins and nutrients which may be used are any orally administrable substances in a suitable formulation. The drinking straw preferably contains a precisely determined dose of these substances, the administration of which is preferably desired as a single dose.

The barrier device preferably takes the form of a plug, the cross-section of which is preferably adapted to the cross-section of the drinking straw. The uninserted plug may, however also have a larger cross-section, provided that this plug can be compressed to a cross-section corresponding to the internal cross-section of the drinking straw.

The barrier device may be permanently fixed in the drinking straw or be arranged movably. The plug may accordingly, on the one hand, be permanently fixed in the drinking straw, by fixing the plug, for example, by narrowing the drinking straw or by adhesively bonding the plug in the drinking straw. The plug is preferably fixed by narrowing the drinking straw.

The plug may, on the other hand, also be movable by the conveying liquid between two limit stops. The drinking straw is narrowed by these limit stops, wherein the narrowed portions are designed such that the plug-like barrier device is retained thereby, but the formulation particles insoluble in the conveying liquid are not.

The plug provided as the barrier device preferably consists of filter material, particularly preferably of a nonwoven material or a compressed fibrous material.

A membrane fixed in the drinking straw is also preferred as a barrier device, wherein the cross-section of the membrane is preferably adapted to the cross-section of the drinking straw and the membrane is preferably adhesively bonded in the drinking straw. The membrane preferably consists of a synthetic material.

Another preferred barrier device is a mesh which is fixed in the drinking straw and has a mesh diameter smaller than the diameter of the smallest formulation particle. The cross-section of the mesh is preferably adapted to the cross-section of the drinking straw and the mesh is preferably fixed in the drinking straw by narrowing of the drinking straw or is adhesively bonded in the drinking straw. The mesh preferably consists of synthetic material.

A narrowing of the drinking straw which is designed such that even the smallest formulation particles are retained thereby is also suitable as a barrier device.

The barrier device must not reduce the flow of the conveying liquid to such an extent that a solid, insoluble active ingredient, vitamin and/or nutrient formulation can no longer be entrained. The barrier device serves in particular as a retaining means for the mobilised formulation, such that the latter is arranged between the mouth orifice and the barrier device.

The drinking straw may be rigid or flexible, straight, preferably reversibly bendable, or angled.

The drinking straw preferably consists of a synthetic material. At least the optionally present zone in which the drinking straw is reversibly bendable is preferably produced from a resilient synthetic material or a synthetic material which is plastically deformable at a temperature below the softening point thereof. The structure of the zone in which the drinking straw is reversibly bendable preferably resembles an accordion.

The internal diameter of the drinking straw should preferably be at least 3 mm such that a sufficient quantity of conveying liquid to convey the formulation can be sucked through the drinking straw. An internal diameter of 4-15 mm is preferred, an internal diameter of 5-10 mm being very particularly preferred.

The drinking straw preferably has a round, oval, rectangular or square cross-section and the inside thereof is preferably smooth.

The drinking straw orifice facing the formulation preferably assumes the form of a mouthpiece, particularly preferably ribbed, while the drinking straw orifice facing the barrier device preferably takes the form of a connecting piece to a reservoir of the conveying liquid. The drinking straw is preferably labelled to indicate the mouth part and/or to indicate the direction of sucking for the conveying liquid. Such marking simplifies handling.

At least the part of the drinking straw containing the formulation is transparent and/or coloured. When a transparent drinking straw is used, uptake of the active ingredient, vitamin and/or nutrient formulation may be observed, while using an opaquely coloured drinking straw makes it possible to conceal the administration of an active ingredient, vitamin and/or nutrient formulation.

The present invention also provides a kit comprising the dosage form according to the invention and a physiologically unproblematic conveying liquid which optionally contains active ingredients, nutrients and/or vitamins.

A suitable conveying liquid is preferably an aqueous liquid, wherein water, lemonade, fruit juice without fruit pulp, tea or coffee are particularly preferred.

The present invention also provides a process for the production of the dosage form according to the invention, in which the barrier device is inserted into the drinking straw, the formulation is introduced into the drinking straw through one drinking straw orifice and the drinking straw orifice facing the formulation is provided with the closing device.

The present invention also provides a process for the production of the dosage form according to the invention in which one drinking straw orifice is provided with the closing device and the formulation and the barrier device are inserted in succession into the drinking straw through the other drinking straw orifice.

A process for the production of the dosage form according to the invention is preferred in which at least one end of the drinking straw is also provided with a removable cap.

The following Figures show exemplary embodiments of the dosage forms according to the invention, but this does not constitute any limitation of the concept of the invention.

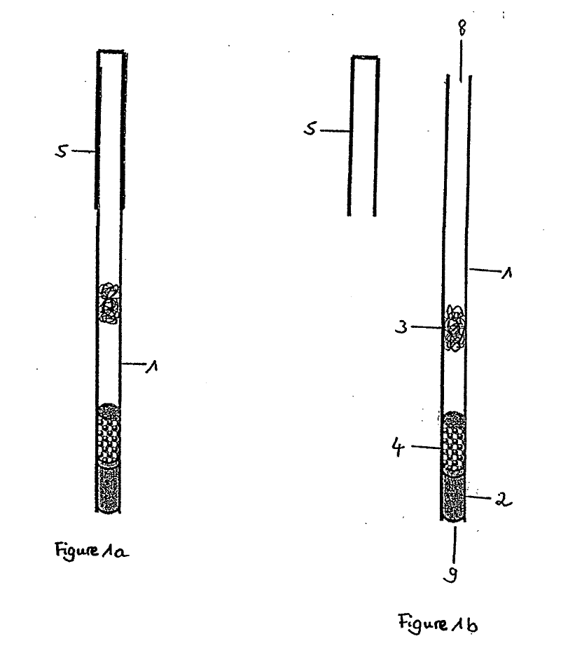

FIGS. 1a and 1b show a side view of a dosage form according to the invention, one with the cap in place (a) and one with the cap removed (b).

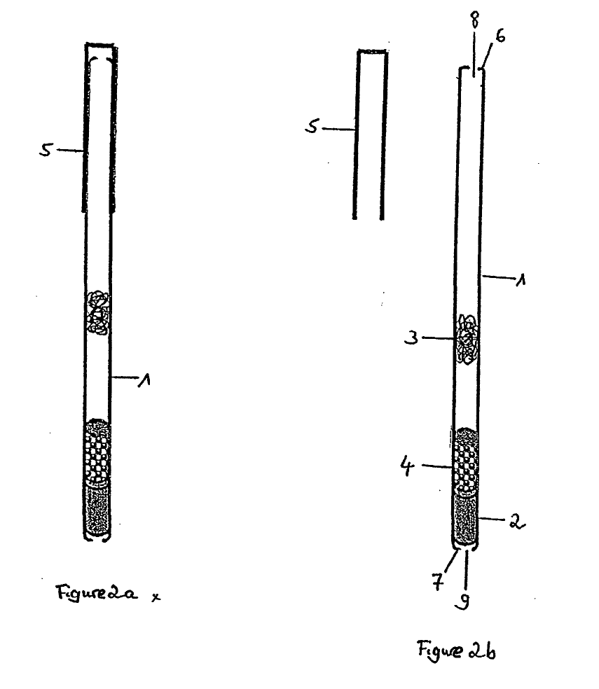

FIGS. 2a and 2b show another side view of a dosage form according to the invention, one with the cap in place (a) and one with the cap removed (b).

The dosage form shown in FIGS. 1a and 1b comprises a drinking straw (1), which has two orifices (8, 9) and a plug fixed in the drinking straw as a barrier device (2), a candyfloss plug as a closing device (3) and a formulation (4) located in the drinking straw between the barrier device and the closing device. The drinking straw (1) may be provided with a cap (5).

The drinking straw (1) has a round cross-section and is straight.

The plug (2) serving as a barrier device consists of filter material and is permeable to air and the conveying liquid, but impermeable to the formulation. The plug is cylindrical and has a round cross-section, wherein the diameter of the plug is adapted to the internal diameter of the drinking straw. The plug is permanently fixed in the drinking straw.

The formulation (4) assumes the form of spherical particles.

The candyfloss plug (3) serving as the closing device is impermeable to the formulation prior to administration, but is permeable to air and soluble in the conveying liquid.

The drinking straw orifice (8) facing the candyfloss plug (3) may be closed with a removable cap (5). The internal diameter of the cap is adapted to the external diameter of the drinking straw (1).

The dosage form shown in FIGS. 2a and 2b substantially corresponds to the dosage form shown in FIGS. 1a and 1b. Instead of a fixed plug, however, a plug which is movable by the conveying liquid between two limit stops (6, 7) is used as the barrier device (2).

The diameter of the largest formulation particles is less than the internal diameter of the drinking straw at the limit stop (6), such that the formulations particles can pass through this point.

1. A dosage form for the oral administration of active ingredients, vitamins and/or nutrients comprising a drinking straw (1), which comprises two orifices (8, 9) and contains a barrier device (2) which is impermeable to an active ingredient, vitamin and/or nutrient formulation, but is permeable to air and a conveying liquid, a closing device (3) which is impermeable to the active ingredient, vitamin and/or nutrient formulation prior to administration, and the active ingredient, vitamin and/or nutrient formulation (4) arranged in the drinking straw between the barrier device and the closing device, characterised in that the closing device (3) is permeable to air and is at least partially soluble in the conveying liquid and consists of a physiologically unproblematic material.

2. A dosage form according to claim 1, characterised in that the closing device is completely soluble in the conveying liquid.

3. A dosage form according to claim 1, characterised in that the closing device (3) has the form of a plug.

4. A dosage form according to claim 1, characterised in that the closing device (3) is a mesh.

5. A dosage form according to claim 1, characterised in that the closing device (3) is a film, preferably a wafer.

6. A dosage form according to claim 1, characterised in that the closing device (3) consist of at least one material or at least one compound selected from among the group comprising sugars and/or sugar-like compounds, preferably sucrose, glucose, fructose, mannitol, sorbitol, flucose, maltose, lactose, galactose, trehalose and/or sugar alcohols; cyclic sugars, preferably cyclodextrins; proteins, preferably gelatine, gelatine derivatives, gelatine hydroxylates; dextrins; proteins from soya, wheat and/or psyllium seeds; gums and/or gum-like materials, preferably agar, acacia, guar, xanthan; flour, preferably rye, wheat and/or rice flour; starch, preferably starch consisting of amylose with up to 6000 glucose units and amylopectin with 10-60 glucose units; modified starch, preferably starch with esterified hydroxy groups, hydroxyethyl starch, hydroxypropyl starch and/or distarch phosphates; pectins; dextrans; cellulose derivatives; preferably methylcellulose, ethylcellulose, hydroxymethylcellulose, hydroxyethylcellulose, carboxymethylcellulose or the alkali metal salts thereof; alginates, carrageenans; amino acid, preferably with 2-12 carbon atoms, particularly preferably glycine, L-alanine, L-aspartic acid, L-glutamic acid, L-hydroxyproline, L-leucine, L-isoleucine and/or L-phenylalanine, synthetic polymers, preferably polyvinylpyrrolidone; inorganic salts, preferably sodium phosphate, sodium chloride and/or aluminium silicate, which optionally also serve as a matrix material for immobilising the formulation.

7. A dosage form according to claim 1, characterised in that at least one drinking straw orifice (8) is provided with a removable cap (5).

8. A dosage form according to claim 1, characterised in that the active ingredient, vitamin and/or nutrient formulation (4) is solid.

9. A dosage form according to claim 1 characterised in that the active ingredient, vitamin and/or nutrient formulation (4) is pasty.

10. A dosage form according to claim 1, characterised in that the active ingredient, vitamin and/or nutrient formulation (4) is in multiparticulate form.

11. A dosage form according to claim 1, characterised in that the active ingredient, vitamin and/or nutrient formulation (4) is insoluble in the conveying liquid.

12. A dosage form according to claim 1, characterised in that the active ingredient, vitamin and/or nutrient formulation (4) is at least partially soluble in the conveying liquid.

13. A dosage form according to claim 1, characterised in that the barrier device (2) is a plug.

14. A dosage form according to claim 13, characterised in that the plug (2) is movable by the conveying liquid between two limit stops (6, 7).

15. A dosage form according to claim 13, characterised in that the plug is fixed.

16. A dosage form according to claim 1, characterised in that the barrier device (2) is a membrane fixed in the drinking straw.

17. A dosage form according to claim 10, characterised in that the barrier device (2) is a mesh fixed in the drinking straw, the mesh diameter of which is smaller than the smallest diameter of the formulation particles (4).

18. A dosage form according to claim 10, characterised in that the barrier device (2) consists of a narrowing of the drinking straw, wherein the cross-section of the drinking straw is narrower than the smallest diameter of the formulation particles (4).

19. A dosage form according to claim 1, characterised in that the drinking straw (1) is rigid or flexible, straight, preferably reversibly bendable, or angled.

20. A kit for the oral administration of active ingredients, vitamins and/or nutrients comprising a drinking straw (1), which comprises two orifices (8, 9) and contains a barrier device (2) which is impermeable to an active ingredient, vitamin and/or nutrient formulation, but is permeable to air and a conveying liquid, a closing device (3) which is impermeable to the active ingredient, vitamin and/or nutrient formulation prior to administration, and the active ingredient, vitamin and/or nutrient formulation (4) arranged in the drinking straw between the barrier device and the closing device, characterised in that the closing device (3) is permeable to air and is at least partially soluble in the conveying liquid and consists of a physiologically unproblematic material.

21. A kit according to claim 20, characterised in that the conveying liquid is an aqueous liquid.

22. A kit according to claim 19, characterised in that the conveying liquid is water, lemonade, fruit juice without the fruit pulp, tea or coffee.
